(19) United States
(12) Patent Application Publication
(10) Pub. No.: US 2022/0378972 A1
(43) Pub. Date: Dec. 1, 2022

(54) UVC DISINFECTION AND FILTRATION SYSTEM AND METHOD OF USE

(71) Applicants: James G. Riesenberger, Canton, GA (US); Ryan J. Riesenberger, Canton, GA (US)

(72) Inventors: James G. Riesenberger, Canton, GA (US); Ryan J. Riesenberger, Canton, GA (US)

(21) Appl. No.: 17/818,574

(22) Filed: Aug. 9, 2022

Related U.S. Application Data
(63) Continuation-in-part of application No. 17/543,142, filed on Dec. 6, 2021.
(60) Provisional application No. 63/230,938, filed on Aug. 9, 2021.
(60) Provisional application No. 63/121,347, filed on Dec. 4, 2020.

(51) Int. Cl. A61L 9/20

(52) U.S. Cl. CPC A61L 9/20; A61L 2209/12; A61L 2209/16

(57) ABSTRACT

A UVC vehicle air disinfection system. Modular and/or self-contained modifications to existing conveyances, vehicles and/or systems to accommodate the housing and electrification of UVC bulbs and a blower within the occupied space of a vehicle to accomplish the task of disinfecting the air within the enclosed-space vehicle during operation without exposing the drivers, passengers, patients, and/or medical personnel to harmful UVC radiation. Components of the disclosed system include an enclosure having an inlet and outlet with UVC bulbs and filtration media therebetween. In use, the UVC bulbs illuminate the enclosure as air passes from the inlet to outlet, inactivating or killing microorganisms contained therein and thereby sterilizing the air of the vehicle.

### CROSS-REFERENCE TO RELATED APPLICATIONS

To the full extent permitted by law, the present United States Non-Provisional Patent Application hereby claims priority to and the full benefit of, United States Provisional Application entitled “UVC AIRSTREAM DISINFECTION FOR AMBULANCE TYPES 1 AND 3,” having assigned Ser. No. 63/230,938, filed on Aug. 9, 2021, and a continuation-in-part of United States Non-Provisional Patent Application entitled “SYSTEM AND METHOD FOR UVC AIRSTREAM DISINFECTION,” having assigned Ser. No. 17/543,142, filed on Dec. 6, 2021, which claims priority to and the full benefit of, United States Provisional Application entitled UVC AIRSTREAM DISINFECTION FOR MACK (GRANITE] TRUCK,” having assigned Ser. No. 63/121,347, filed on Dec. 4, 2020, which are incorporated herein by reference in their entirety.

### FEDERALLY SPONSORED RESEARCH OR DEVELOPMENT

None

### PARTIES TO A JOINT RESEARCH AGREEMENT

None

### REFERENCE TO A SEQUENCE LISTING

None

### BACKGROUND OF THE DISCLOSURE

### Technical Field of the Disclosure

The instant disclosure relates to air disinfection and filtration, namely passenger and commercial vehicle air disinfection and filtration. More particularly, the instant disclosure relates to the manufacture and installation of an improved air handling accessory to introduce disinfecting UVC light therein.

### Description of the Related Art

Improvements in air handling designs have improved overall indoor and vehicle air quality, primarily through implementation of increases in ventilation and air filtration technology. Nevertheless, new building construction and vehicles, including passenger cars, trucks, vans, etc. as well as buses, tractor trailers, fire trucks, ambulances, etc. may fail to filter airborne microscopic viral and bacterial organisms due to disparities particle size versus filter pore size. Commonly, air filter pore size is much larger than viral and other microscopic organisms. Additionally, filtering systems having very small pores sufficient to suspend airborne organisms may significantly impact overall airflow through a building HVAC and/or vehicle airstream system. This often means a trade-off between volume/rate of airflow and filtration ability which can inevitably cause inefficiencies in an airstream system. Installation of even very high-filtration systems thus decreases the overall rate at which air may pass through a system, which may then be compensated for by running a system at a higher fan speed. Nevertheless, even through use of very fine filters, small organisms and/or the bioaerosol droplets in which they may be suspended, may still pass through such a filter and remain suspended within a vehicle compartment until it is either ingested (breathed or otherwise deposited on human tissue), deposited on a surface, exited via ventilation, or caught in a second pass through the filter. The ideal or the best indoor air quality filtration systems, such as those found in state-of-the-art hospitals, usually house HEPA filters which may be rated at 0.3-1 microns (MERV 17-20). Minimum Efficient Reporting Value (MERV) may be used to quantify and/or rate this tradeoff between filtering power and efficiency. Filters having high MERV rating and small particulate-size capturing capabilities, usually eliminate, or filter out over 99% of airborne particulates smaller than this size, which should capture many airborne droplets greater than this size. However, if the organisms themselves are smaller or dry out and become part of a smaller airborne particle, it may pass through or around such filtering media as bypass air.

In response to the recent emergence of SARS-CoV-2 as an infectious cause of COVID-19, many infectious disease experts have developed measures to contain and/or limit its spread among individuals as they interact indoors with one another. These have involved widespread adoption of universal masking policies indoors, vaccination campaigns and/or mandates, surveillance testing for the virus's presence, social/physical distancing measures, routinized handwashing, other personal protective equipment (PPE), and other restrictions and/or counter measures seeking to limit the spread of SARS-CoV-2 and its variants among the global population. Each has achieved some level of spread-reduction, though the global pandemic continues to affect many individuals' daily lives through such restrictions and attempts to contain the virus. Air quality experts in particular have offered multiple solutions, both previously-known and novel, in order to limit the spread of the virus indoors. These include but are not limited to increasing the ventilation of indoor spaces (i.e., bringing in air from outside), chemical or ion air treatments, and UVC air treatment. Each implementation brings with it various tradeoffs, much like the restrictions and prevention measures adopted and/or imposed on individuals. Increasing ventilation in climate-controlled spaces by definition causes inefficiencies due to the need to heat and/or cool outside air to maintain a comfortable indoor temperature. Since outdoor air is generally thought to be free of viral infectious organisms, this tradeoff has been readily adapted despite the related inefficiencies. However, other tradeoffs may exist with this strategy including environmental air contamination (especially in urban areas), allergen introduction which may require additional filtration, and heating, ventilation, and air-conditioning (HVAC) system wear. UVC (ultraviolet radiation) air treatment has been adopted by schools, hospitals, businesses, and other building types as a way to inactivate/eliminate pathogens in the air non-specifically. Once a pathogen is inactivated or sterilized, it cannot reproduce and the air may be considered disinfected.

All radiation is a form of energy, most of which is invisible to the human eye. UV radiation is only one form of radiation and it is measured on a scientific scale called the electromagnetic (EM) spectrum. UV radiation is a type of EM energy along a specific band of the EM spectrum. Radio waves are EM waves which transmit sound from a radio station's tower to, for instance a stereo, or between cell phones. Microwaves, like those that heat food in a microwave oven are another type of EM radiation as well as visible light that is emitted from the lights in a home. UV radiation, specifically, is the portion of the EM spectrum between X-rays and visible light. This UV spectrum is broken into at least three types: UVA, UVB, and UVC. UVA rays have the longest wavelength, followed by UVB and UVC, which has the shortest. All three are emitted by Earth's Sun, but only UVA and UVB are thought to reach Earth's surface. UVC radiation from the sun does not reach Earth's surface because it is blocked by the ozone layer of the Earth's atmosphere. Thus, the only way humans can be exposed to UVC radiation (at least while on Earth), is through an artificial source, such as a lamp or laser having UVC-emission properties. Like all UV radiation, UVC radiation can cause mild and even severe burns of the skin and eye injuries (photokeratitis). Individuals are advised to avoid direct skin exposure to UVC radiation and never look directly into a UVC light source, even briefly. Skin burns and eye injuries from UVC exposure usually resolve within a week with no known long-term damage. Due to the short wavelength of UVC, the penetration depth of UVC radiation is very low, and the risk of skin cancer, cataracts or permanent vision loss is also thought to be very low. The type of eye injury associated with exposure to UVC causes severe pain and a feeling of having sand in the eyes. Sometimes people are unable to use their eyes for one to two days. It can occur after a very short exposure (seconds to minutes) to UVC radiation.

UVC radiation, specifically, is a known disinfectant for air, water, and nonporous surfaces. UVC radiation has effectively been used for decades to reduce the spread of bacteria, such as tuberculosis. For this reason, UVC lamps are often called “germicidal” lamps. Prior to the emergence of SARS-CoV-2, hospitals may have been the primary adoptees of this technology with respect to air handling systems. This could prevent infection in surgical settings, protect immunocompromised individuals, and limit transmission of infectious agents among patients, staff, and visitors in hospitals. Due to the concerns regarding human safety, utilization of UVC treatment may involve installation and/or use in an unoccupied space where a UVC bulb may illuminate the space with UVC radiation during periods the room is unoccupied (e.g., an office at night) in order to sterilize both the air and surfaces in contact with the UVC radiation or installation within an HVAC system airstream disinfection, which would not be occupied by individuals within any particular building and can be used to treat air as it passes out of occupied space and into the system. In the case of the former, treatment of occupied space, the installation may be permanent with occupancy sensors or may be temporary and portable, which may be rolled into a room during a period of unoccupancy. During maintenance, installation, and other activities requiring individuals to access the UVC lamps, they may require protective equipment and/or a simple switch to deactivate during a potential exposure.

Specific to SARS-CoV-2, UVC radiation has been shown to destroy the outer protein coating of other individual SARS-Coronavirus organisms, which may be different virus from the current SARS-CoV-2 virus. Other studies show UVC's ability to create one or more dimers along the DNA or RNA strands of viruses, or otherwise affect their genetic material, causing the virus's replication capabilities to be interfered with, thereby stopping transmission. Many promising studies, investigations, and installations have yielded indication that this specific virus is no different than previously studied coronaviruses in this regard. The destruction of the outer protein coating and/or dimerization (or other radiation-caused damage) of its genetic material ultimately leads to inactivation of the virus. When viewed under a microscope, observers often remark that the organisms look like they have been “cooked” or “fried”. However, currently there is limited published data about the wavelength, dose, and duration of UVC radiation required to inactivate the SARS-CoV-2 virus. It is thought and/or hypothesized that other mechanisms of interaction between the viral organism(s) and UVC radiation may similarly kill, disinfect, and/or neutralize the virus in question. These include but are not limited to effects on viral DNA/RNA, viral proteins (i.e., non-coating proteins such as the SARS-CoV-2 Si spike protein and its variants), and/or other important viral cellular features which impact their virality and/or infectiousness.

In the past, vehicle airstream and/or vehicle HVAC systems were uncommonly candidates for installation of UVC or other air disinfection systems. Though reasons may vary, this may have been due to their expense, size, as well as the fact that most vehicles have limited occupancy. Due to the emergence of SARS-CoV-2 as a global infectious agent which has caused a pandemic, vehicle air quality has received renewed interest. Where previously, air systems which relied on heavy filtration of outside air in order to prevent introduction of pollutants and/or allergens into the vehicle cabin, interest has grown into the direction if increasing the ventilation and/or filtering recirculated air (rather than outdoor air being filtered upon introduction into the vehicle). While filtration of incoming air may still be important to vehicle occupant health, vehicle occupant safety now deserves a new focus—the disinfection of recirculated air, particularly during winter and summer operation. The challenges of fitting lamps capable of sufficiently “dosing” and/or fluence possibly contaminated air with UVC, as well as other challenges related to retrofitting existing vehicle HVAC systems with such lamps has remained a challenge. This and the risk-factors noted above may be especially true for vehicles which carry multiple occupants simultaneously. Even more so in vehicles which are designed to be driven to a destination, or series of destinations, to pick up an occupant and deliver them to a destination. Examples include but are not limited to buses, shuttles, taxis/limousines or other hired vehicles, trains and their passenger cars, ambulances, firetrucks, ferries, cable cars, enclosed lifts, the like and/or combinations thereof. No known solution offers a ready-made solution for these common vehicle types which may be efficiently and economically run, though the need to protect certain individuals from being infected remains. In particular, drivers of vehicles which are involved in the transport of individuals, as well as the passengers thereof, are highly critical to the maintenance of modern commerce and transport. Protection of those individuals from infection may be key to sustaining our modern economy, supply chain, lifestyle, and confidence thereof.

Therefore, it is readily apparent that there is a recognized unmet need for a system and method for disinfecting vehicle air through use of UVC lamps. The instant disclosure is designed to address this need through installation of an apparatus within a vehicle HVAC system and methods of installation and use, which includes the systems, apparatuses, and methods disclosed herein while addressing at least some of the aspects of the problems discussed above.

### SUMMARY

Briefly described, in a possibly preferred embodiment, the present disclosure overcomes the above-mentioned disadvantages and meets the recognized need for such an UVC vehicle disinfection system and method of installation and use. The primary apparatus may involve one or more UVC lamps installed or simply placed within a vehicle. Additional modifications may be required, depending on the size and configuration of specific vehicles. For instance, larger vehicles and those having multiple compartments may require multiple installations/placements. Additionally, access to these systems may require security such that passengers or other unauthorized individuals do not access internal features of such a device so as to prevent damage to the device and prevent injury to those accessing them. Furthermore, sufficient power to the UVC lamps must be supplied in order to ensure sufficient power to operate during transit. Many other modifications may be required, which may involve the conversion of power, safety (from radiation and electrical shock), ease of installation, ease of maintenance, and other bulb protection/stability considerations.

More specifically, highly specific example embodiments of the present system and method may be disclosed herein. Namely, the assembly of, in a potentially preferred embodiment, of a serviceable and/or portable device and/or apparatus such that a new air handling apparatus(es) and/or components thereof are provided in order to allow for a turn-key device to disinfect vehicle air. This may include but is not limited to new power supplies/sources, new ballasts, new mounts/securing apparatuses, new wiring harnesses, new converters or inverters, new filtration media, new safety switches, new status and maintenance alerting features, and new enclosures may be required for UVC installations in commercial trucking vehicles. Additionally, in a potentially preferred embodiment, safety switches may be activated upon opening such an apparatus and/or device and special treatments may be applied to filtration media thereof. More specifically, these new modifications/installations may include those related to ambulances, firetrucks, trains, buses, shuttles, and other vehicles commonly involved in commercial transpiration. Additionally, these vehicles may be customized and/or specially fit to accommodate such a device of the disclosure. While modification of existing vehicles in commercial fleets may be a subject of this disclosure, original equipment may sufficiently accommodate at the time of initial manufacture to include the disclosed components, thereby making these features and components compatible with original equipment manufacturer (OEM) parts. Additionally, these features and components, including the device(s) of the disclosure, may be offered as standard equipment, optional OEM equipment, or dealer-installed and/or aftermarket equipment, as may be understood by those having ordinary skill in the art.

The devices, apparatuses, systems, and methods of the disclosure may provide various features and benefits, as will be understood by those having ordinary skill in the art. These include but are not limited to disinfection of vehicle interiors at a specified airflow rate, simple/inexpensive/quick installation/maintenance/service, low-power consumption, limited vehicle performance reduction, limited and/or non-existent airflow reduction, increased airflow, sufficient UVC pathogen exposure time, multiple interior cabin air turnovers per hour, the like and/or combinations thereof.

In a possibly preferred exemplary embodiment, the devices, apparatuses, systems, and methods disclosed herein may relate specifically to ambulances and firetrucks as well as other vehicle systems manufactured by other manufacturers which may share some or many features with such vehicles. Additionally, the modifications, devices, apparatuses, and systems of the disclosure may specifically include but are not limited to UVC lamp(s), transformers, converters, inverters, ballast(s), interconnecting wiring, wiring harness(es) (e.g., Molex® type connectors, wire-to-wire connectors), and air handling enclosures and the modification thereof. Additional modifications and installations may include additional holes/apertures, compartments, vents, ducts, and support brackets to accommodate the other modifications and installations.

In another possibly preferred exemplary embodiment, the devices, apparatuses, systems, and methods disclosed herein may relate to a portable and/or modular device manufactured to be placed and/or installed in an optimal compartment and/or area of a vehicle. In such an embodiment, the devices, apparatuses, systems, and methods may include features such as customizable or customized: air intake(s)/vents/ducts having one or more openings in order to optimize air flow via intake, air return(s)/vents/ducts having one or more openings in order to optimize air flow via outlet, replaceable filtration media, status-indicating lights, safety switches activated upon opening, the like and/or combinations thereof. Other embodiments may include features and/or components which may monitor various other features and/or components and communicate statuses of the same, by way of example and not limitation may include operating parameters such as run time, on/off statuses, bulb status, filter status, ballast status, pressure drop(s) across the system(s), the like and/or combinations thereof, and monitoring such statuses via a remote computer, such as may be available via cloud computing technologies.

These and other features of the disclosed disinfection system and methods of installation and operation will become more apparent to one skilled in the art from the prior Summary and following Brief Description of the Drawings, Detailed Description of exemplary embodiments thereof, and Claims when read in light of the accompanying Drawings or Figures.

### BRIEF DESCRIPTION OF THE DRAWINGS

The disclosed apparatus and methods of use will be better understood by reading the Detailed Description with reference to the accompanying drawings, which are not necessarily drawn to scale, and in which like reference numerals denote similar structure and refer to like elements throughout, and in which:

FIG. 1 is a plan view of an exemplary embodiment of the system of the disclosure with a side removed to expose the internal components thereof;

FIG. 2 is a plan view of the embodiment featuring an optional safety mechanism;

FIG. 3 is a diagram showing exemplary vehicle placements of the system; and

FIG. 4 is a flow diagram of an exemplary method of installation and use.

It is to be noted that the drawings presented are intended solely for the purpose of illustration and that they are, therefore, neither desired nor intended to limit the disclosure to any or all of the exact details of construction shown, except insofar as they may be deemed essential to the claimed disclosure.

### DETAILED DESCRIPTION

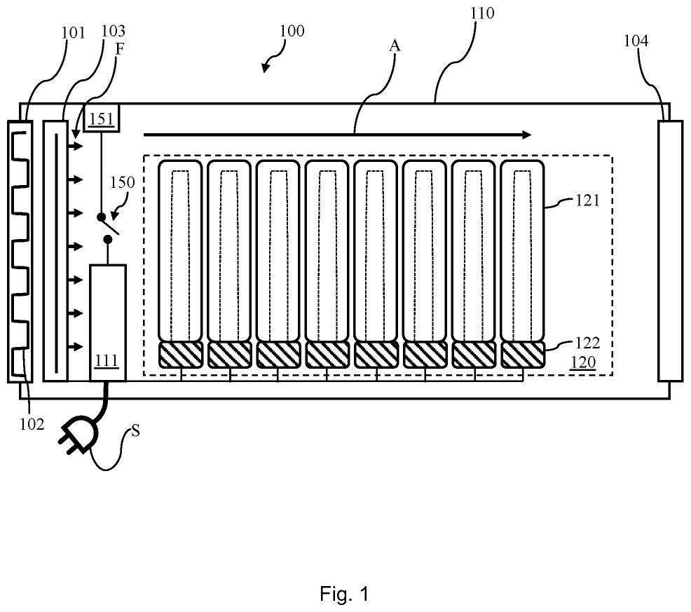

In describing the exemplary embodiments of the present disclosure, as illustrated in FIGS. 1-4, specific terminology is employed for the sake of clarity. The present disclosure, however, is not intended to be limited to the specific terminology so selected, and it is to be understood that each specific element includes all technical equivalents that operate in a similar manner to accomplish similar functions. Embodiments of the claims may, however, be embodied in many different forms and should not be construed to be limited to the embodiments set forth herein. The examples set forth herein are non-limiting examples, and are merely examples among other possible examples. It should be noted that the term vehicle and/or automobile may be used herein as a descriptor for any motorized means of transportation. Additionally, the system and methods of the disclosure may be widely applicable to any occupied indoor space, whether as part of a conveyance, or as part of a fixed construction, such as a building or room. The term disinfection as used herein shall mean any reduction in microorganism population within air through any means. Microorganisms shall mean any virus and/or multi- or uni-cellular organism or group of such organisms of small size and generally capable of replication within or about a larger lifeform to cause an infection or other undesirable characteristic thereof. The terms UV, UVC, and radiation, the like, and combinations thereof may be used to refer to any illumination, though invisible, which may introduce electromagnetic radiation into air. The terms airstream, HVAC, ventilation, and vehicle air handling system may be used interchangeably to mean systems which are designed to move air within an indoor and/or vehicle or other occupied space. Additionally, various components and/or steps of the disclosed system, apparatuses, devices, and methods may be described in various ways. By way of example and not limitation, a void through which air passes through such a system may be referred to as a compartment, enclosure, light reactor, clamshell enclosure, airstream, duct, vent, or simply void. Power, electricity, and other terms which refer specifically to the transmission of electrons to power electrical devices may be used herein to describe various modifications and installations of the disclosed system, apparatuses, devices, and the methods of installation, operation, use, and maintenance thereof.

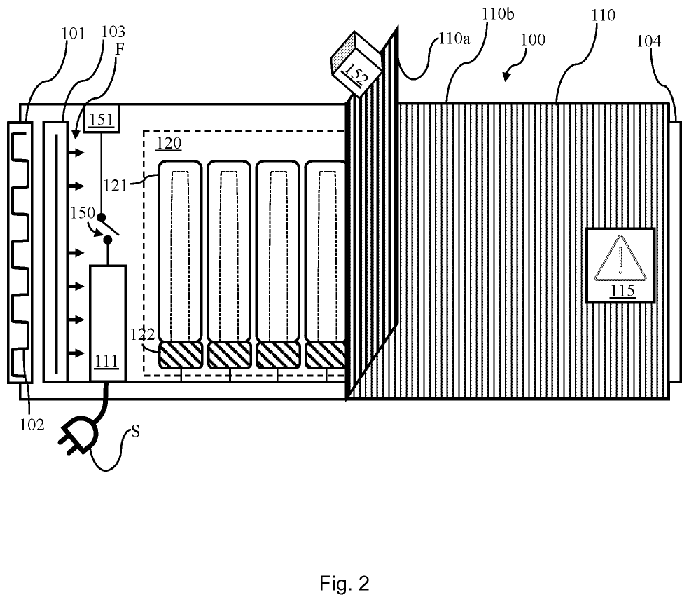

Referring now to specifically FIGS. 1-2 by way of example, and not limitation, therein illustrated are plan views of an exemplary embodiment of the system of the disclosure. Though some components may be illustrated therein to have a shape, size, and spatial relationship, the disclosure is not so limited. Illustrative and artistic choices therein are used solely for recognition purposes, including symbols and icons which may represent features and/or components of a vehicles, enclosures, air handling systems, and/or electronic components thereof which may or may not be a feature of the system of the disclosure. As may be used herein, the term “bulb(s)” may be understood to mean any device(s) or apparatus(es) capable of illuminating a physical space upon electrification and may include other terms known to those having ordinary skill in the art, including but not limited to “lamp(s)”. Furthermore, the term “blower(s)” may be understood to mean any device(s), apparatus(es), and/or system(s) capable of causing a variation in air pressure over said device(s), apparatus(es), and/or system(s), and may further include such terms as “fan(s)”.

Referring specifically to FIG. 1, therein illustrated is a plan view of an exemplary embodiment of UVC air sanitizing system 100 of the disclosure. In order to show the internal features of UVC air sanitizing system 100, various features, such as first top cover 110a and second top cover 110b may not be shown. UVC air sanitizing system 100 may be powered by power source S via power adapter 111. Power source S and power adapter 111 may be adapted and/or interchangeable based upon the power needs of UVC air sanitizing system 100 and/or conveyance V and the internal electrical power abilities thereof. In a possibly preferred embodiment which may be adapted specifically to conveyance V which may be the industry standard Type 1 and 3 ambulances, power adapter 111 may be a 500 W inverter having 12V DC and 120V AC compatibility. Perhaps importantly, various safety features may exist within UVC air sanitizing system 100 in order to terminate power to the various systems and components found in UVC air sanitizing system 100. As illustrated herein, this may include interruption switch 150, which may be operably combined with body sensor 151, as may be covered in greater detail in FIG. 2 and its related Detailed Description. The electronic parts of UVC air sanitizing system 100 may be supplied using this exemplary electrical power system, or may not be supplied power if such power would be unsafe or otherwise deleterious to health, as may be understood by those having ordinary skill in the art.

Turning now to the various air-handling features of UVC air sanitizing system 100 as may be illustrated therein FIG. 1, UVC air sanitizing system 100 may feature air intake vent 101 having disposed thereon and/or therein filtration media 102. Power may be supplied via power source S and power adapter 111 to blower 103, which may be a fan or similar device capable of moving air through a system such as UVC air sanitizing system 100. Airflow may generally follow airflow direction A, though as those having ordinary skill in the art may understand, various obstacles and the general behavior of air and airborne material may be more complex. This may mean that airflow direction A is simply an air pressure gradient or general/average direction of gas and airborne material/suspended particles through a system such as UVC air sanitizing system 100. Airflow direction A may be caused by, for instance, spinning air turbulation of blower 103 in fan direction F, and may feature reversibility to change the direction of airflow direction A, simply by reversing polarity, as may be understood by those having ordinary skill in the art. As illustrated herein, air and any unfiltered suspended particulate material may exit through supply outlet 104 into the occupied space(s) of conveyance V. While the disclosure herein has provided one arrangement of air intake vent 101, filtration media 102, blower 103, and supply outlet 104, but the disclosure is not limited to this arrangement. Those having ordinary skill in the art may understand that multiple air intake vents 101, filtration media 102, blowers 103, and/or air returns 104 may be provided in order to increase airflow and/or pressure within UVC air sanitizing system 100, or a change in arrangement may be necessary in order to accommodate certain conveyances V. By way of example and not limitation, this order may be reversed, filtration media 102 may be placed proximate supply outlet 104, air intake vent 101 may be placed on a bottom/top surface of UVC air sanitizing system 100, or other modifications to UVC air sanitizing system 100 in order to direct and handle airflow direction A and thereby modify the use and compatibility of UVC air sanitizing system 100. As may be understood by those having ordinary skill in the art, airflow direction A may sustain through enclosure 110 from air intake vent 101 to supply outlet 104. In at least one preferred embodiment of the disclosed UVC air sanitizing system 100, UVC air sanitizing system 100 may measure approximately 40 in. from air intake vent 101 to supply outlet 104 and approximately 10.4 in. along the sides containing either of air intake vent 101 and supply outlet 104.

In a potentially preferred embodiment of the air handling components of UVC air sanitizing system 100, these components may have specific qualities and/or characteristics which are herein described by way of example and not limitation. blower 103 may be a fan, such as a 3 in. EC centrifugal fan having 120 CFM capability and further having, in such a preferred embodiment, compatibility with 120V A/C, 30 W, and 0.5 A power demands/capabilities. Filtration media 102 may measure 5.5 in. high by 10 in. wide by 2 in. deep and may be a pleated MERV 11 with 120 CFM capabilities at 250 FPM and filter particle sizes at 85 percent efficiency on particles that are 1.0 to 10 micrometers in size. Additionally, as may be found in a potentially preferred embodiment, such filtration media 102 may be specially treated with protective material(s) in order to prevent degradation subsequent prolonged exposure to UVC and other illumination sources as may be found in UVC air sanitizing system 100. Additionally, in other preferred embodiments, filtration media 102 may be shielded from such UVC and other radiation to similar ends. These may include non- and/or semi-transparent materials and may further require that, in order to prevent airflow obstruction, they be used in combination with radiation-absorbing coatings and/or materials to prevent radiation reflection within UVC air sanitizing system 100, or may be otherwise shielded from UVC radiation therein UVC air sanitizing system 100. As those having ordinary skill in the art may understand, pigments and/or colorations of the internal components of UVC air sanitizing system 100, including filtration media 102 itself, may assist in prevention of such degradation. As may be understood by those having ordinary skill in the art, UVC shielding and/or coatings may bring with them various tradeoffs, including but not limited to additional expense in manufacture and reduced airflow of UVC air sanitizing system 100.

Turning now to the illuminating and disinfecting features herein disclosed of UVC air sanitizing system 100, UVC air sanitizing system 100 may feature bulb array 120. Each bulb 121 of bulb array 120 may feature socket 122 which may obtain necessary power from power source S and power adapter 111. Though eight (8) bulbs 121, each having socket 122, are illustrated herein, the disclosure is not so limited. Each bulb 121 may feature its own socket 122 or one socket 122 may feature multiple bulbs 121. The reverse may be true as well. In a potentially preferred embodiment of UVC air sanitizing system 100, eight (8) PHILLIPS® Model TUV PL-L 35 W/4p HO 11 W UVC Output 42V 0.8 A bulbs may be installed therein enclosure 110 of UVC air sanitizing system 100, along with the necessary and/or optional features to power such an arrangement, as may be disclosed herein. The illuminating and/or disinfecting features, such as bulb array 120 may further feature ballasts which may be featured in interceding operable combination therebetween power source S and/or power adapter 111 and bulb 121 and/or socket 122. Such ballasts may share compatibility with the bulb/socket combination along with the power source S and power adapter 111, as may be understood by those having ordinary skill in the art. In a potentially preferred embodiment, four (4) ballasts may be used to each accommodate two (2) bulb/socket combinations for a total of eight (8) bulbs 121 within UVC air sanitizing system 100 and such ballasts may each possess a program start feature, such as may be found in FULLHAM® Model SHS 11-UNV-H having 1.39 A and 120V power requirements.

Turning now to FIG. 2, therein illustrated is a plan view of an exemplary embodiment of UVC air sanitizing system 100 of the disclosure. Instead of removing a top side of enclosure 110 as may be illustrated in FIG. 1, the top side is shown in a possibly hinged and open embodiment to illustrate a potential safety feature thereof such a potentially preferred embodiment. Enclosure 110, featuring first top cover 110a and second top cover 110b may have each of first top cover 110a and second top cover 110b in operable combination with enclosure 110 such that they may be removably attached and/or hinged. Additionally, first top cover 110a and/or second top cover 110b may each contain warning label 115 in order to advise/warn conveyance V operators and/or their passengers that UVC bulbs may be in operation, which may be deleterious to human health via radiation. Additional safety features as illustrated therein FIG. 2 may include, for instance body sensor 151 and lid sensor 152, which may be used in operable combination with interruption switch 150 and cause electrification or terminate electrification of UVC air sanitizing system 100 and/or the various electrical components thereof. It is further contemplated herein that body sensor 151 and lid sensor 152, in combination may instead form a latch, which also forms a circuit, the completion of which may activate UVC air sanitizing system 100 or its component parts (e.g., bulb array 120), removing the need of a separate safety mechanism and/or switch, such as interruption switch 150. As may be understood by those having ordinary skill in the art, body sensor 151 and lid sensor 152 may be specially configured to sense when enclosure 110 is opened at, for instance first top cover 110a, and cause interruption switch 150 to suspend power to bulbs 121 via the electrical system(s) thereof UVC air sanitizing system 100. Additionally, other features receiving power may similarly be interrupted via interruption switch 150, thereby causing UVC air sanitizing system 100 to variously power down during maintenance, installation, and/or inadvertent opening in order to prevent deleterious and/or dangerous consequences to drivers, passengers, operators, maintenance personnel, or other human contacts with conveyance V and/or UVC air sanitizing system 100. Other features illustrated in FIG. 2 include those presented in FIG. 1 and described therein, including air intake vent 101, filtration media 102, blower 103, power source S, power adapter 111, bulb 121, socket 122, and bulb array 120.

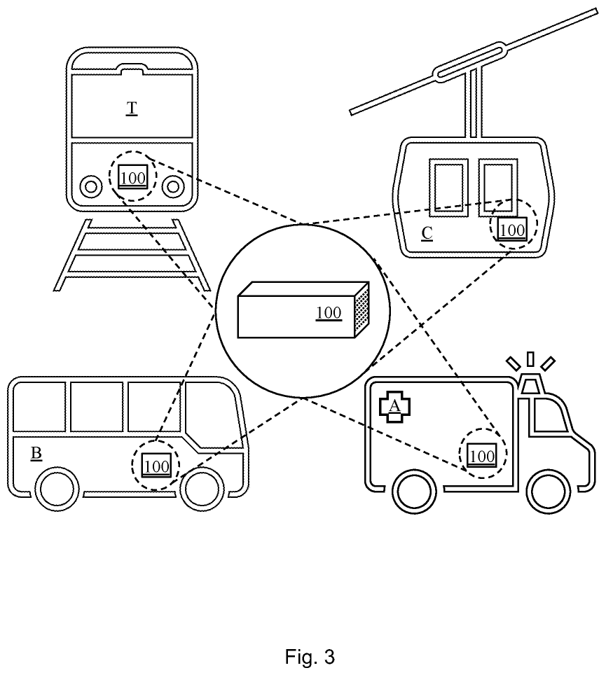

Turning now to FIG. 3, therein illustrated are various exemplary conveyances V, which may feature UVC air sanitizing system 100. As illustrated therein, in potentially preferred embodiments of UVC air sanitizing system 100, UVC air sanitizing system 100 may be relatively compact to fit within various conveyances V, such as passenger rail car T, cable car C, bus B, and ambulance A. As may be understood by those having ordinary skill in the art, placement of UVC air sanitizing system 100 may be key to ensuring airflow is not obstructed such that occupied spaces and the gasses and suspended materials thereof are captured via air intake vent 101 through UVC air sanitizing system 100 and out supply outlet 104. Additionally, multiple UVC air sanitizing systems 100 of the disclosure may be present, as is illustrated by way of example and not limitation within bus B. As may be understood by those having skill in the art, should multiple occupants occupy a large space within conveyance V, such as with large passenger busses B, multiple UVC air sanitizing systems 100 may be necessary to sufficiently treat the air within such occupied spaces in order to ensure the cubic footage thereof is sufficiently disinfected. Additionally, even smaller occupied spaces, such as ambulance A, may feature multiple UVC air sanitizing systems 100 in order to treat various compartments or occupied spaces which may contain infected individuals, as may be the case when an ambulance is transiting a person suffering extreme consequences of infection, which may induce coughing, sneezing, or other exhalants that may be capable of infecting uninfected passengers/drivers of conveyance V. By way of example and not limitation, as it may relate to ambulance A, the patient/treating professional occupied space of ambulance A may feature one UVC air sanitizing system 100, and the driver occupied space may be physically separated and further include another UVC air sanitizing system 100, such that an unknown infection of a passenger/driver within the driver occupied space is less likely to infect the other occupant of the space. Many other potentially preferred embodiments and conveyances V may exist and operate in combination with UVC air sanitizing system 100, and are intended to be encompassed by the disclosure herein. These include but are not limited to buses, shuttles, taxis/limousines or other hired/for-hire vehicles, trains and their passenger cars, ambulances, firetrucks, ferries, cable cars, enclosed lifts, the like and/or combinations thereof.

Having full view of the various components of the system of the disclosure as illustrated therein FIGS. 1-3, one having ordinary skill in the art can appreciate various features and options to determine the best configuration for any given vehicle. Given that a space at or UVC air sanitizing system 100 may be very close to other features and equipment, causing a very tight or cramped space there around UVC air sanitizing system 100, electrical components of the system of the disclosure may be housed within UVC air sanitizing system 100, or may alternatively be housed remotely within the vehicle, and be powered and controlled via a wiring or other remotely controlled and/or powered design. Additional modifications, such as those herein described, may be required to ensure sufficient stabilization for the components and/or features of UVC air sanitizing system 100. Such modifications may be made to UVC air sanitizing system 100 and the features thereof. Additionally, brackets, mounts, gaskets, encapsulating socket(s), and other stabilizing apparatuses which may or may not stabilize at one, two, or more areas within and/or about enclosure 110 may be added to UVC air sanitizing system 100. In various preferred embodiments of the disclosed system, UVC air sanitizing system 100 may be suitably placed at or near the cooling coil within a conveyance V HVAC system, which may allow full airflow from the discharge of blower 103, or may turn the stream of air, for instance, by 90 degrees before entering the coil. Since bulb array 120 may only project radiation into the interior of UVC air sanitizing system 100, where the air is directed, the forced air through airstream enclosure and past bulb array 120, sufficient time may pass for the sterilizing features of UVC radiation to have its effect upon those microbial air aspects necessary to sufficiently sterilize the air. Furthermore, such sterilization may not require filtration at all, though filtration media 102 may accomplish two separate benefits. Namely, these may include preventing buildup of dropped airborne particles within UVC air sanitizing system 100 and suspending them on filtration media 102 in order to prevent them from reentering occupied space within conveyance V. This aspect may be important to ensuring that not only is UVC air sanitizing system 100 relatively free of particles which may interfere with the various systems and components thereof, but that bulb array 120 and/or each bulb 121 operate with minimal contamination/residue, which may interfere with and/or reduce the UVC radiation which emits therefrom, thereby reducing the overall disinfecting power of UVC air sanitizing system 100. Also particularly important may be a design which ensures that any airborne organism, (such as a pathogen, virus, etc.) cannot pass through UVC air sanitizing system 100 at a distance greater than 1½ inches from a bulb 121 of bulb array 120 in order to ensure inactivation/death of these airborne organisms during their transit through UVC air sanitizing system 100. Enclosure 110 of UVC air sanitizing system 100 may be manufactured of various materials, some of which may be disclosed herein. These may include aluminum, stainless steel, other metals, plastics, wood, other natural/synthetic materials, the like and/or combinations thereof. These materials may be chosen and/or specially manufactured or treated to facilitate reflection and/or absorption of radiation within and/or from UVC air sanitizing system 100. By way of example and not limitation, aluminum/stainless steel may be highly polished or otherwise treated to increase reflection within UVC air sanitizing system 100 in order to increase exposure of airborne microorganisms to the UVC radiation therein, and thereby facilitating their inactivation/death. A maximum kill rate of the pathogens may be accomplished through use of other materials in combination with bulb array 120 and enclosure 110. Other materials and/or treatments may be used to prevent escape of radiation from UVC air sanitizing system 100 and are herein contemplated. In optional embodiments of UVC air sanitizing system 100, enclosure 110 may be modular, such that various panels and/or sides may be exchanged and/or removably attached so as to allow for varying configurations of UVC air sanitizing system 100. By way of example and not limitation, a first side may be originally configured as a single solid side, but later exchanged for a side having, for example air intake vent 101 or supply outlet 104. Alternatively, air intake vent 101 and supply outlet 104 may be swapped in order to reverse the filtration media 102 thereof air intake vent 101. While air intake vent 101 is illustrated and described as having filtration media 102 thereon/thereof, the disclosure is not so limited. UVC air sanitizing system 100 may feature no filtration media 102 or many, and may be configured to receive standard and/or custom filtration media 102, which may be washed, cleaned, laundered, and/or exchanged, depending on the various benefits and tradeoffs thereof.

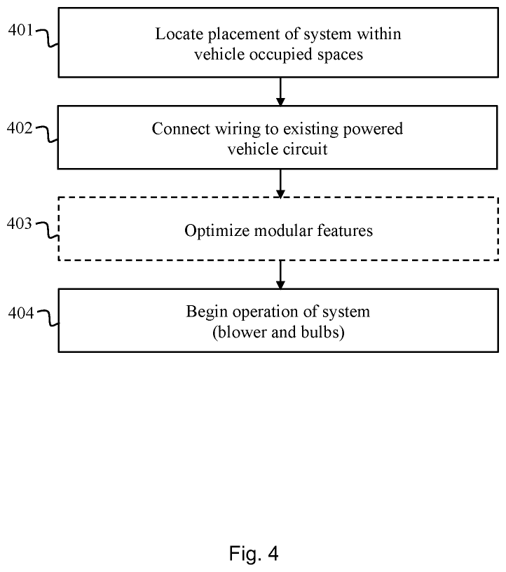

Referring specifically to FIG. 4, therein illustrated is a flow diagram of an exemplary method of installation and use. As described herein, few modifications may be required to existing conveyances V in order to provide disinfecting power via UVC air sanitizing system 100. Ideally, only a void within conveyance V having access to occupied space within conveyance V, and the power necessary to electrify UVC air sanitizing system 100 may be necessary. At method step 401, such a space within conveyance V is located and UVC air sanitizing system 100 is connected to an existing circuit of the electrical system thereof conveyance V at method step 402. Optionally, at optional step 403, if modular components of enclosure 110 are provided, as described herein, airflow direction and air intake vent 101 and supply outlet 104 may be optimized for a given conveyance V. Limited experimentation may be necessary, which may include monitoring airflow at air intake vent 101 and/or supply outlet 104. In a potentially preferred embodiment, at optional step 403, such airflow may be detected by UVC air sanitizing system 100 itself to achieve optimal and/or suitable results. Having found an optimal and/or suitable location and configuration, the method may proceed to method step 404, which may cause UVC air sanitizing system 100 to become electrified via power source S, and the various components of UVC air sanitizing system 100 may then receive power to bring air into UVC air sanitizing system 100 via air intake vent 101, through filtration media, using blower 103, and the air may be sterilized by bulb array 120 and exit UVC air sanitizing system 100 through supply outlet 104. Operation of UVC air sanitizing system 100 may then proceed to consistently sterilize the air within conveyance V, such that its occupants are prevented from infecting one another, thereby continuously reducing pathogen concentration within the occupied space(s) of conveyance V during operation of UVC air sanitizing system 100.

The illustrations and diagrams illustrated and described herein are intended to provide a general understanding of the structure of various embodiments of the disclosed system and method of vehicle airstream disinfection. The illustrations are not intended to serve as a complete description of all of the elements and features of the apparatus, product, method of use, and/or system that utilizes the structures and/or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

With respect to the above description then, it is to be realized that the optimum dimensional relationships, to include variations in size, materials, shape, form, position, function and manner of operation, assembly, type of airflow mechanism, power supply, type of bulb(s), shape of housing(s) enclosure(s), material(s) of housing(s), material(s) of enclosure(s), mounting location(s), placement location(s) vehicle type and use, all of which are intended to be encompassed by the present disclosure.

It is contemplated herein that the device may include a variety of overall sizes and corresponding sizes for and of various parts, including but not limited to: UVC lamps, transformer(s), converter(s), ballast(s), interconnecting wiring, relay(s), switch(es), electrical connections (e.g., MOLEX® or similar type connectors), and electrical enclosures. Furthermore, it is contemplated that due to variations in vehicles, their size and shape, their onboard power systems, their existing ventilation and/or HVAC systems, including but not limited to passenger cars, passenger trucks, cable cars, buses, ferries, ships, boats, train cars, locomotives, submersibles, aircraft, the like and/or any combination of enclosed transport thereof, adjustments may be made to the disclosed device to better adapt the system of the disclosure to the intended enclosed transport vehicle. Yet still, though the inventor has contemplated one method of sterilizing the air contained in enclosed passenger transport vehicles and the like, the disclosure is not limited to specific UVC lights, transformer(s), converter(s) ballast(s), interconnecting wiring, relay(s), switch(es), electrical connections (e.g., MOLEX® or similar type connectors), potentiometers, and electrical enclosures. Other means that have been contemplated and which the disclosed system and method may include, but the disclosure is not so limited, variations in air sterilization methods (e.g. other UV wavelengths, heat, steam, pressure, ionization), variations in amperage and voltage of transformers/inverters/converters (possibly through use of potentiometers), variation in size/shape/material for ballast, variation in lamps/bulbs to include but not limited to e.g., LED, variation in wiring and corresponding connections/harnesses, as well as size, shape, and material for construction of enclosure 110. Various trade-offs may be considered when selecting the technology to deploy in the systems and methods of the disclosure. These include limiting the power consumption during use or idle, installing an on/off switch, including new methods of air sterilization as they are developed and/or discovered, including filtration devices within the system to collect airborne particles subsequent and/or prior to UV treatment, sensors and/or indicators to determine operability, air quality, light or UVC radiation intensity during operation, and level of operation, and/or status lights and/or interconnectivity of the device to third party devices via a variety of technologies including but not limited to: onboard vehicle systems, onboard vehicle diagnostic systems/computers (e.g. OBD), wired or wireless computing devices that may connect to such systems or to the device itself, cloud systems that may remotely connect and monitor the device through long-range wireless technologies (e.g. 3G or LTE). It is also contemplated that certain considerations and/or additional features of the present disclosure may improve the functionality. These may include communication protocols to alert a status of bulb 121 or bulb array 120 health, filtration media 102 life and airflow quality, cleaning and sanitization protocols, and the like and combinations thereof. In regard to communication with other devices via a network, the device may communicate via any known or yet to be discovered protocol, including wired networking, fiber optic communication, wireless networking (i.e. WiFi), near field communication (e.g. Bluetooth® or NFC), the like or combinations thereof. The device may receive power from the vehicle in which it is designed to be installed, may have a universal power adapter, may be designed to operably combine with a vehicle cigarette lighter socket, a separate power source external and/or temporary to the vehicle (e.g., when a driver exits a vehicle and a new driver enters or when certain seats of a bus B are occupied), a solar and/or wind energy source installed onto the vehicle, or may contain a battery which may or may not have the capability to re-charge.

The foregoing description and drawings comprise illustrative embodiments of the present disclosure. Having thus described exemplary embodiments, it should be noted by those ordinarily skilled in the art that the within disclosures are exemplary only, and that various other alternatives, adaptations, and modifications may be made within the scope of the present disclosure. Merely listing or numbering the steps of a method in a certain order does not constitute any limitation on the order of the steps of that method. Many modifications and other embodiments of the disclosure will come to mind to one ordinarily skilled in the art to which this disclosure pertains having the benefit of the teachings presented in the foregoing descriptions and the associated drawings. Although specific terms may be employed herein, they are used in a generic and descriptive sense only and not for purposes of limitation. Moreover, the present disclosure has been described in detail, it should be understood that various changes, substitutions and alterations can be made thereto without departing from the spirit and scope of the disclosure as defined by the appended claims. Accordingly, the present disclosure is not limited to the specific embodiments illustrated herein, but is limited only by the following claims.

What is claimed is:

1. A UVC air sanitizing system for a vehicle having a power source comprising:
a power adapter capable of receiving power from the power source;
an enclosure having an air intake vent and an air supply;
a UVC bulb array within said enclosure and in operable combination to the power adapter, said UVC bulb array disposed between said air intake vent and said air supply;
a blower in operable combination to the power adapter proximate said air intake vent and within said enclosure; and
a filtration medium therein said enclosure.

2. The system of claim 1, wherein the vehicle is selected from a group and the group consists of a bus, a passenger shuttle, a taxi, a limousine, a ridesharing vehicle, a train, an ambulance, a firetruck, a ferry, a cable car, and an elevator.

3. The system of claim 1, wherein said UVC bulb array is eight UVC bulbs.

4. The system of claim 3, wherein each of said UVC bulb array is in a plurality of four bulb pairs, each of said plurality of four bulb pairs is connected to one of a plurality of ballasts.

5. The system of claim 4, wherein said plurality of ballasts is four ballasts.

6. The system of claim 1, wherein said blower is a fan.

7. The system of claim 1, wherein said enclosure further comprises a service panel, said service panel is configured in an open position and a closed position.

8. The system of claim 7, further comprising an interruption switch disposed between the power source and said UVC bulb array.

9. The system of claim 8, wherein said interruption switch terminates power to said UVC bulb array when said service panel is in said open position.

10. The system of claim 1, wherein said filtration medium is proximate said air intake.

11. The system of claim 2, wherein said filtration medium is physically shielded from a radiation of said UVC bulb array.

12. A method of continuously disinfecting an air of a vehicle cabin having a power source, the method comprising:
placing a UVC air sanitizing system therein the vehicle cabin, the UVC air sanitizing system having a power adapter capable of receiving power from the power source, an enclosure having an air intake vent and an air supply, therebetween said air intake vent and said air return are a UVC bulb array, a blower and a filtration medium;
electrifying the UVC air sanitizing system by operably combining the power source with said power adapter; and
disinfecting the air of the vehicle cabin.

13. The method of claim 12, wherein the vehicle is selected from a group and the group consists of a bus, a passenger shuttle, a taxi, a limousine, a ridesharing vehicle, a train, an ambulance, a firetruck, a ferry, a cable car, and an elevator.

14. The method of claim 12, wherein said UVC bulb array is eight UVC bulbs.

15. The method of claim 12, wherein said blower is a fan.

16. The system of claim 12, wherein said enclosure further comprises a service panel, said service panel is configured in an open position and a closed position.

17. The system of claim 16, further comprising an interruption switch disposed between the power source and said UVC bulb array.

18. The system of claim 17, wherein said interruption switch terminates power to said UVC bulb array when said service panel is in said open position.

19. The system of claim 12, wherein said filtration medium is proximate said air intake.

20. The system of claim 19, wherein said filtration medium is physically shielded from a radiation of said UVC bulb array.
